(19) United States
(12) Patent Application Publication
(10) Pub. No.: US 2025/0152609 A1
(43) Pub. Date: May 15, 2025

(54) SALICYLIC ACID COMPOSITIONS AND USES THEREOF

(71) Applicants: PHARMALP SA, CONTHEY (CH); UNIVERSITÄT BASEL, BASEL (CH)

(72) Inventors: PHILIPPE MEUWLY, AUBONNE (CH); OLIVIER POTTERAT, AESCH (CH); BRUNO SCHNYDER, CRANS-MONTANA (CH)

(21) Appl. No.: 18/840,197

(22) PCT Filed: Feb. 22, 2023
(86) PCT No.: PCT/EP2023/054364

§ 371 Date: Aug. 21, 2024

(30) Foreign Application Priority Data
Feb. 22, 2022 (EP) 22158048.3

(51) Int. Cl. A61K 31/60; A61K 9/00; A61K 31/593; A61K 31/7004; A61K 31/7048; A61K 36/73; A61P 31/04

(52) U.S. Cl. CPC A61K 31/60; A61K 9/0053; A61K 31/593; A61K 31/7004; A61K 31/7048; A61K 36/73; A61P 31/04

(57) ABSTRACT

The present invention is directed to compounds, compositions, uses and methods useful in the prevention or treatment of urinary infections, in particular E. coli infections and in particular recurrent urinary infections.

### FIELD OF THE INVENTION

The present invention relates to prevention or treatment of urinary infections, including E. coli infections and in particular to compositions useful for the prevention or treatment of urinary infections.

### BACKGROUND OF THE INVENTION

Urinary tract infection (UTI) is one of the most common infection affecting millions of people every year. Women are particularly affected, since they have a 50% risk of suffering from this disease during their life (5% in men) and UTI accounts for more than 1 million hospitalizations and $1.6 billion in medical expenses each year in the USA (Ulett et al., 2013, Cur Opin Microbiol., 16: 100-107). More than half of the women experience a relapse of the infection within 6 months and 70 to 95% of reported cases of UTI are primary caused by the uro-pathogenic Escherichia coli (E. coli) (Foxman 2002, Am J Med., 113, Suppl. 1A:5S-13S). Infection occurs through the following steps: E. coli interacts with uro-epithelium via bacterial pili I adhesion. Epithelial defense molecules are released. However, invasion into the host cell protects E. coli from the defense and allows its replication. A further defense is the detachment of epithelial cells infected with E. coli for clearance of the infection via urine excretion. The defense is at the cost of the host cells and is termed “exfoliation”. In case of incomplete clearance, E. coli persists in fibrous form, which is highly infectious and protected from phagocytosis. UTI requires antimicrobial treatments, such as antibiotics, which often result in the emergence of resistant microbial flora and new approaches for prevention and treatment of UTI are therefore needed (Hooton et al., 2004, Clin Infect Dis. 39(1): 75-80).

Symptoms of uncomplicated UTIs are pain on urination (dysuria), frequent urination (frequency), inability to start the urine stream (hesitancy), sudden onset of the need to urinate (urgency), and blood in the urine (hematuria).

Phenolic acids are typically divided into hydroxybenzoic acids and hydroxycinnamic acids (Valanciene et al., 2020, Biomolecules, 10, 874). Salicylic acid (SA) and its derivatives such as salicylic acid methyl ester and monotropitoside (methylsalicylate O-beta-D-xylopyranosyl-(1->6)-O-beta-D-glucopyranoside) belong to hydroxybenzoic acids and derivatives of those. Salicylic acid (SA) serves as a key hormone in plant innate immunity, including resistance in both local and systemic tissue upon biotic attacks, hypersensitive responses, and cell death (Ding et al., 2020, Trends Plant Sci. 25(6), 549-565). Salicylates have shown a broad spectrum of biological activities, including antithrombotic, anti-inflammatory, anti-neoplastic and anti-microbial properties (Wu, 2007, Anti-Inflam Anti-Allerg Agents Medicin Chem, 6, 278).

Oenothein B is an ellagitannin dimer with a macrocyclic structure which is present in medicinal Oenothera, Epilobium, and Eucalyptus species and has been found having various biological activities (Yoshida et al., Molecules 2018, 23(3), 552).

There are important needs for new strategies of prevention and/or treatment of urinary tract infections, in particular E. coli infections. In particular, multi-targeting effects are desirable in the antimicrobial area since targeting a single enzyme by anti-infectious drugs may trigger resistance mechanisms.

### SUMMARY OF THE INVENTION

The present invention relates to the unexpected finding that salicylic acid and some of its metabolites formed in vivo after ingestion of salicylic acid have a beneficial activity against E. coli bacteria adhesion on bladder cells but also a bactericidal and diuretic effect that is beneficial in the prevention and/or treatment against UTI, in particular E. coli infections. Further surprisingly, it was found that oenothein B and extracts of Epilobium parviflorum containing oenothein B play a role on the inhibition on E. coli growth in vivo that would be complementary to the activity of salicylic acids and some of its metabolites and advantageous in the prevention and/or treatment of a UTI, in particular an E. coli infection.

A first aspect of the invention provides salicylic acid (SA) or a metabolite thereof or a mixture thereof or pharmaceutical compositions thereof for use by oral route in the prevention and/or treatment of a urinary tract infection, wherein said metabolite is a metabolite formed in vivo after ingestion of salicylic acid.

According to another further aspect, the invention provides plants of the species Filipendula ulmaria and/or parts of these plants and/or extracts of these plants or pharmaceutical compositions thereof for use by oral route according to the present invention in the prevention and/or treatment of a urinary tract infection.

In another embodiment of the invention, is provided a use of salicylic acid (SA) or a metabolite thereof, or a mixture thereof or pharmaceutical compositions thereof, for the preparation of an oral pharmaceutical composition for the prevention, repression and/or treatment of a urinary infection.

In another embodiment of the invention, the invention relates to a formulation comprising from about 1 to about 550 mg, for example from 10 to about 200 mg dry matter weight of a Filipendula ulmaria extract and at least one pharmaceutically or cosmetically acceptable carrier, diluent or excipient thereof, in particular for use for the prevention, repression and/or treatment of a urinary infection.

In another embodiment of the invention, the invention relates to a formulation comprising salicylic acid (SA) or a metabolite thereof, or a mixture thereof or pharmaceutical compositions thereof, combined with at least one co-agent useful in the prevention and/or treatment of urinary infections and at least one pharmaceutically or cosmetically acceptable carrier, diluent or excipient thereof.

In another embodiment of the invention, the invention relates to a formulation comprising an extract of Filipendula ulmaria, combined with at least one co-agent useful in the prevention and/or treatment of urinary infections and at least one pharmaceutically or cosmetically acceptable carrier, diluent or excipient thereof.

In another embodiment of the invention is provided a method for preventing and/or treating a urinary infection in a subject, said method comprising administering by oral route in a subject in need thereof an effective amount of salicylic acid (SA) or a metabolite thereof, or a mixture thereof or pharmaceutical compositions thereof.

In another embodiment of the invention is provided a method for preventing and/or treating a urinary infection in a subject, said method comprising administering by oral route in a subject in need thereof an effective amount of Filipendula ulmaria extract or a formulation thereof.

### DESCRIPTION OF THE FIGURES

FIG. 1 shows the effects on the adhesion of E. coli to human urinary bladder cells, quantifying the number of adhered living bacteria by plate-count-agar culturing (cfu) of compounds and extracts of the invention as described in Examples 1 to 3. Inhibitory effects are indicated relative to negative control with no inhibitory substance (=100%, on y-axis). Cranberry juice extract is used as a control, as it is known to have a beneficial effect in vivo. Effects of Filipendula ulmaria extracts (A) and of Epilobium parviflorum extracts (B) wherein extracts were extracted by aqueous (0% ethanol) (1), 20% ethanol (2), and 70% ethanol (3) solutions; C: Salicylic acid (SA) (1) and two metabolites thereof, i.e. 2,3-dihydroxybenzoic acid (2) and salicyluric acid (3; D: Mixtures of extracts from Filipendula ulmaria (F) and from Epilobium parviflorum (E); E: representation of the anti-adhesion activity of each of the extracts of the invention taken alone ((E) at 2.5 mg/mL; (F) at 2.5 mg/mL) and in combination as a mixture of the invention ((30% (E)+70% (F) at a total of 5 mg/mL), compared to cranberry extracts (C) (2.5 mg/mL) and D-mannose (D) (2.5 mg/ml); F: representation of the prevention of growth of E. coli (MIC) of each of the extracts or the substances alone ((F) at 0.9 mg/mL; (E) at 2.7 mg/mL; Oenothein B (O) at 1.3 mg/mL; DHBA (2) at 2.5 mg/mL; SUA (3) at 2.5 mg/mL; SA (1) at 5 mg/mL) compared to cranberry extracts (C) (25 mg/mL).

FIG. 2 represents the phytochemical characterization of the extracts as described in Example 4. A: HPLC profile of an extract of Filipendula ulmaria and the detection of salicylic acid (t_{R} 19.8 min) by HPLC-UV analysis. A SunFire C₁₈ column (3.5 μm, 3.0×150 mm) equipped with a guard column (3.0×10.0 mm) was used. The mobile phase consisted of H₂O (A) and MeCN (B), both containing 0.2% formic acid. A gradient of 15-40% B for 30 min, 40-100% B for 1 min, and 100% B for 10.00 min was applied at a flow rate of 0.4 mL/min; B: Chromatographic profile of Epilobium parviflorum extract and detection of oenothein B. A SunFire C₁₈ column (3.5 μm, 3.0×150 mm) equipped with a guard column (3.0×10.0 mm) was used. The mobile phase consisted of H₂O (A) and MeCN (B), both containing 0.1% formic acid. A gradient of 5-50% B in 30 min, was applied at a flow rate of 0.4 mL/min. Peaks 1-3: oenothein B, a myricetin-hexoside and hyperoside, respectively. ELSD: evaporative light scattering detection.

FIG. 3 shows the detection and the content of salicylic acid and its metabolites in urine samples collected from mice treated with Filipendula ulmaria extract as described in Example 3. A: Comparison of RT from analytical standards of salicylic acid (SA) (1) and its metabolites 3-dihydroxybenzoic acid (DHBA) (2) and salicyluric acid (SUA) (3) and peaks found in urine samples collected from the group treated with Filipendula ulmaria extract (Fil). Analysis was performed on a SunFire™ C18 (3.5 m, 3.0 mm×150 mm) column (Waters). 5% MeCN (A) and MeCN (B) both containing 0.1% of formic acid were used as mobile phase. A gradient of 0 to 100% B in 20 min was applied at a flow rate of 0.8 mL/min. B: Concentration, amounts (in samples collected over a 3-hour-period) and cumulative amounts of salicylic acid (SA) (1) and its metabolites 2,3-dihydroxybenzoic acid (DHBA) (2) and salicyluric acid (SUA) (3) found in urine samples collected from groups treated with Filipendula ulmaria extract (F). Cumulative amounts are the cumulated amounts of the respective compounds in the urine over time up to the indicated timepoint. Numbers on x-axis indicate collection time. Analysis was performed by HPLC-MRM-MS using the chromatographic conditions described in A.

FIG. 4 represents the amount of urine collected for 24 hours using a mouse model and a combination of extracts according to the invention compared to controls as described in Example 3.

FIG. 5 shows the in vivo anti-adhesion effect on E. coli of substances and extracts of the invention in a mouse model described as described in Example expressed in Colony Forming Units (cfu) and in percent of variation compared to control. Each value has been calculated from a total of 6 mice. 10⁶ means 1′000′000 cfu.

FIG. 6 presents the results of the tests of efficacy in women with recurrent UTIs for the extract mixtures as described in Example 6. A: Improvement of urinary symptoms in patients in absence of treatment (light grey) compared to patients treated with a combination of extracts (dark grey); B: Mean duration of symptoms of urinary infections (days) in absence of treatment (light grey) compared to patients treated with a combination of extracts (dark grey); C: percentage of satisfaction among treated patients.

### DETAILED DESCRIPTION

The term “pharmaceutically acceptable” refers to a carrier comprised of a material that is not biologically or otherwise undesirable.

The term “cosmetically acceptable” refers to a carrier comprised of a material that is not biologically or otherwise undesirable for topical use on the skin or mucosa.

The term “carrier” refers to any components present in a pharmaceutical formulation other than the active agent and thus includes diluents, binders, lubricants, disintegrants, fillers, coloring agents, wetting or emulsifying agents, pH buffering agents, preservatives and the like.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and is not necessarily meant to imply cure or complete abolition of symptoms, but refers to any type of treatment that imparts a benefit to a patient and includes: (a) preventing the disease from occurring in a subject which may be predisposed to the disease but has not yet been diagnosed as having it for example based on familial history, overweight status or age; (b) inhibiting the disease, i.e. arresting its development; or relieving the disease, i.e. causing regression of the disease and/or its symptoms or conditions such as improvement or remediation of damage.

In particular, prevention and/or treatment of urinary tract infections according to the invention comprise decreasing sensitivity of an individual to E. coli infections. For example, it comprises bacteriostatic (prevent microbial growth), anti-adhesion and diuretic effects. The term “treatment” refers to any type of treatment or prevention that imparts a benefit to a subject afflicted with or at risk of developing urinary tract infections, including improvement in the condition of the subject (e.g., in one or more physiological conditions) or slowing the progression of infection, etc.

According to one aspect, effects of a treatment or application according to the invention may be observed through the decrease of urinary infection symptoms such as pain or burning sensation when urinating (dysuria), need to urinate more often than usual (pollakiuria), abdominal pain and hematuria.

The term “efficacy” of a treatment, application or method according to the invention can be measured based on the number of E. coli measured in bladder samples. A culture medium is inoculated with the sample which is then incubated at 37° C. for one or several days, to allow development of bacterial colonies. The characteristics, such as number, morphology and colors, of the colonies allow diagnosis of E. coli infections.

### Use According to the Invention

According to a further aspect, the invention provides salicylic acid (SA) or a metabolite thereof or pharmaceutical compositions thereof for use by oral route in the prevention and/or treatment of a urinary tract infection, wherein said SA metabolite is selected from 2,3-dihydroxybenzoic acid and salicyluric acid.

According to a further aspect, SA or a metabolite thereof for use according to the invention wherein SA or a metabolite thereof is provided in a composition comprising from about 0.75 μg to 2.6 mg of SA.

According to a further aspect, is provided an oral formulation comprising salicylic acid (SA) or a metabolite thereof or a mixture thereof, wherein said formulation comprises from about 0.75 μg to 2.6 mg of SA and at least one pharmaceutically or cosmetically acceptable carrier, diluent or excipient thereof, wherein said metabolite is a metabolite formed in vivo after ingestion of salicylic acid, for use for the prevention, repression and/or treatment of a urinary infection.

According to a further aspect, SA is provided in the form of an extract of Filipendula ulmaria.

According to another further aspect, the invention provides plants of the species Filipendula ulmaria and/or parts of these plants and/or extracts of these plants or pharmaceutical compositions thereof for use by oral route in the prevention and/or treatment of a urinary tract infection.

According to another further aspect, the Filipendula ulmaria extract is an extract from the floral tops of Filipendula ulmaria.

According to another further aspect, the Filipendula ulmaria extract is an alcoholic or hydroalcoholic extract.

According to another further aspect, the Filipendula ulmaria extract is an aqueous extract. In another embodiment of the invention, is provided a use of salicylic acid (SA) or a metabolite thereof or pharmaceutical compositions thereof, for the preparation of a pharmaceutical composition for the prevention, repression and/or treatment of a urinary infection by oral route.

Another aspect of the invention relates a use of plants of the species Filipendula ulmaria and/or parts of these plants and/or extracts of these plants or pharmaceutical compositions thereof for the preparation of a herbal medicine for prevention and/or treatment of a urinary tract infection by oral route.

According to a further aspect, the invention provides salicylic acid (SA) or a metabolite thereof or pharmaceutical compositions thereof for use in the prevention and/or treatment of a urinary tract infection by oral route, wherein SA or said SA metabolite is to be used in combination with oenothein B.

According to a further aspect, oenothein B is provided in a composition comprising from about 15 μg to about 65 mg of oenothein B.

According to a further aspect, oenothein B can be provided in the form of an extract of Epilobium, in particular Epilobium parviflorum.

According to another further aspect, the invention provides plants of the species Filipendula ulmaria and/or parts of these plants and/or extracts of these plants or pharmaceutical compositions thereof for use in the prevention and/or treatment of a urinary tract infection by oral route, wherein said plants of the species Filipendula ulmaria and/or parts of these plants and/or extracts of these plants or pharmaceutical compositions thereof are to be administered in combination with plants of the species Epilobium parviflorum and/or parts of these plants and/or extracts of these plants or pharmaceutical compositions thereof

According to another further aspect, the Epilobium parviflorum extract is an extract from the aerial parts of Epilobium parviflorum.

According to another further aspect, the Epilobium parviflorum extract is from Epilobium parviflorum leaves.

According to another further aspect, the Epilobium parviflorum extract is hot water extract.

According to another further aspect, the Epilobium parviflorum extract is an alcoholic or hydroalcoholic extract.

According to another further aspect, the Epilobium parviflorum extract is an aqueous extract.

In another embodiment of the invention is provided a use of a formulation as described herein for the preparation of an oral pharmaceutical composition for the prevention, repression and/or treatment of a urinary infection.

In another embodiment of the invention is provided a method for preventing and/or treating a urinary infection in a subject, said method comprising administering by oral route in a subject in need thereof an effective amount of salicylic acid (SA) or a metabolite thereof or pharmaceutical compositions thereof, wherein said SA metabolite is selected from 2,3-dihydroxybenzoic acid and salicyluric acid.

In another embodiment of the invention is provided a method according to the invention, wherein said salicylic acid (SA) or a metabolite thereof or pharmaceutical compositions thereof is to be administered in combination with oenothein B.

In another embodiment of the invention is provided a method for preventing and/or treating a urinary infection in a subject, said method comprising administering by oral route in a subject in need thereof an effective amount of Filipendula ulmaria extract or a formulation thereof.

In another embodiment of the invention is provided a method according to the invention, wherein said Filipendula ulmaria extract is to be administered in combination with an Epilobium parviflorum extract or a formulation thereof.

In another embodiment of the invention is provided a method according to the invention wherein a formulation as described herein is administered.

### Extracts

The extracts of plants according to the invention are prepared by standard methods, including maceration for 1 h at 70° C. using a material to water ratio of 1/10 followed by percolation, filtration and concentration.

In a particular embodiment, a Filipendula ulmaria extract according to the invention contains from about 0.1 to about 2.5% w/w, for example from about 0.5 to about 2% w/w such as from about 1 to about 1.5% w/w of salicylic acid (SA) or a metabolite thereof.

In a particular embodiment, a therapeutically or prophylactically effective daily dose of an extract of Filipendula ulmaria may be, in humans based on the active doses in mouse, from about 1 to about 550 mg, from about 1 to about 200 mg dry matter weight, in particular from about 1 about 100 mg.

In a further particular embodiment, a therapeutically or prophylactically effective daily dose of an extract of Filipendula ulmaria may be, in humans based on the active doses in mouse, from about 10 to about 200 mg dry matter weight, in particular from about 20 about 100 mg.

In a particular embodiment, a therapeutically or prophylactically effective daily dose of an extract of Epilobium parviflorum may be, in humans based on the active doses in mouse, from about 10 to about 1000 mg dry matter weight, in particular from about 10 to 500 mg.

In a further particular embodiment, a therapeutically or prophylactically effective daily dose of an extract of Epilobium parviflorum may be, in humans based on the active doses in mouse, from about 15 to about 1000 mg dry matter weight, in particular from about 15 to 500 mg.

In another further particular embodiment, a therapeutically or prophylactically effective daily dose of an extract of Epilobium parviflorum may be, in humans based on the active doses in mouse, from about 50 to about 1000 mg dry matter weight, in particular from about 100 to about 500 mg.

In a particular embodiment, a Epilobium parviflorum according to the invention contains from about 0.2 to about 20% w/w, for example from about 1 to about 18% w/w, such as from about 5 to about 15% w/w of oenothein B.

In a particular embodiment, a Epilobium parviflorum according to the invention contains from about 0.5 to about 20% w/w, for example from about 1 to about 18% w/w, such as from about 5 to about 15% w/w of oenothein B.

The extracts may be characterized by the percentage of dry matter which they contain. Dry matter is the solid residue remaining after removal of the carrier or solvent by drying, such as by drying a solution or suspension in an oven, by lyophilization or evaporation under reduced pressure. Dry matter may be expressed in % and may also be referred to as plant solids concentration. Thus, a 100 g or 100 mL solution or suspension containing 5% dry matter by weight, yields 5 grams of solids or residue after drying. Alternative methods of drying may yield slightly different values for the percent by weight of dry matter, so that all such values recited herein are necessarily approximations. Dry matter values include suspended as well as settled solids. The dry matter values of the extracts of the invention have been determined according to The European Pharmacopoeia 8^{th} edition, chapter 2.8.17.

### Compositions

According to an embodiment, is provided a formulation comprising from about 10 to about 200 mg dry matter weight of a Filipendula ulmaria extract and at least one pharmaceutically or cosmetically acceptable carrier, diluent or excipient thereof, in particular useful for the prevention and/or treatment of urinary infections.

According to another embodiment, the invention relates to an oral formulation comprising salicylic acid (SA) or a metabolite thereof or pharmaceutical compositions thereof, combined with at least one co-agent useful in the prevention and/or treatment of urinary infections and at least one pharmaceutically or cosmetically acceptable carrier, diluent or excipient thereof.

In another embodiment of the invention, the invention relates to a formulation comprising an extract of Filipendula ulmaria, combined with at least one co-agent useful in the prevention and/or treatment of urinary infections and at least one pharmaceutically or cosmetically acceptable carrier, diluent or excipient thereof.

According to a particular embodiment, said co-agent is selected from oenothein B and Spiraeoside or a mixture thereof.

According to another particular embodiment, said co-agent is an extract of Epilobium parviflorum.

According to an embodiment, is provided a formulation comprising a combination of an extract of Filipendula ulmaria and of an extract of Epilobium parviflorum. Typically, a combined formulation according to the invention comprises from about 30 to about 70% w/w of an extract of Filipendula ulmaria and from about 70 to about 30% w/w of an extract of Epilobium parviflorum. According to a further particular embodiment, a combined formulation according to the invention comprises from about 30 to about 50% w/w of an extract of Filipendula ulmaria and from about 70 to about 50% w/w of an extract of Epilobium parviflorum.

According to another embodiment, a combined formulation according to the invention comprises from about 15 to about 85% w/w of an extract of Filipendula ulmaria and from about 85 to about 15% w/w of an extract of Epilobium parviflorum. According to a further particular embodiment, a combined formulation according to the invention comprises from about 15 to about 50% w/w of an extract of Filipendula ulmaria and from about 85 to about 50% w/w of an extract of Epilobium parviflorum.

According to an embodiment, is provided a formulation comprising a combination from about 10 to about 200 mg dry matter weight of a Filipendula ulmaria extract with combination from about 10 to about 200 mg dry matter weight of a Epilobium parviflorum extract and at least one pharmaceutically or cosmetically acceptable carrier, diluent or excipient thereof.

According to an embodiment, is provided an oral formulation comprising wherein said formulation comprises from about 1 to about 550 mg dry matter weight of a Filipendula ulmaria extract (e.g. from 10 to about 200 mg) and at least one pharmaceutically or cosmetically acceptable carrier, diluent or excipient thereof.

According to a further particular embodiment is provided an oral formulation comprising 1 to about 550 mg dry matter weight of a Filipendula ulmaria extract (e.g. about 20-25 mg), about 10 to 500 mg of a Epilobium parviflorum extract (e.g. about 100-110 mg) and at least one pharmaceutically or cosmetically acceptable carrier, diluent or excipient thereof.

According to a particular embodiment, the formulation of the invention is for oral use under solid form such as for example tablets (e.g. hard or soft), capsules or powder, under semi-liquid form such as orally drinkable gels and under liquid form such as drinks and syrups.

Compounds, extracts, uses and methods according to the invention are particularly useful in the prevention of urinary infections, in particular E. coli infections. Those present not only the advantage to prevent growth of the bacteria but also to prevent its adhesion to the urinary tract cells which is mostly responsible of recurrence. In addition, uses and methods according to the invention provide the advantage to avoid the use of antibiotic treatments which increase the risk of resistance to pathogenic bacteria.

In another particular embodiment, the invention provides a composition according to the invention for use in the preparation of a food supplement, a cosmetic formulation and a medicament.

According to a particular embodiment, a composition of the invention is a pharmaceutical composition.

According to a particular embodiment, a composition of the invention is a food supplement.

According to a particular embodiment, a composition of the invention is a cosmetic composition.

According to a particular embodiment, a composition of the invention is an oral composition.

Compositions of this invention may further comprise one or more pharmaceutically acceptable additional ingredient(s) such as alum, stabilizers, antimicrobial agents, buffers, coloring agents, flavoring agents, adjuvants, and the like.

The compositions according to the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for topical use.

According to the invention, the compositions of the invention may be prepared in the form of food supplement, medical device, cosmetic products and drug product.

Compositions of this invention may be liquid formulations including, but not limited to, aqueous or oily suspensions, solutions, emulsions. The compositions may also be formulated as a dry product for reconstitution with water or other suitable vehicle before use. Such liquid preparations may contain additives including, but not limited to, suspending agents, emulsifying agents, non-aqueous vehicles and preservatives.

Suspending, dispersing, wetting and emulsifying agents include, but are not limited to polyethyleneglycol, glycerol stearate and sorbitol esters.

Further materials as well as formulation processing techniques and the like are set out in Remington's “The Science and Practice of Pharmacy”, 23^{rd} Edition, 2020, Editor: Adeboye Adejare, Academic Press the content of which is incorporated herein by reference.

### Mode of Administration

Extracts, compounds or compositions of this invention may be administered orally. The exact dose of extracts and compositions is readily determined by one of skill in the art based on the teachings herein, along with the potency of the extract and composition, the age, weight, sex and physiological condition of the subject.

According to one embodiment, compounds or compositions of the invention are applied or administered before or at the beginning of the onset of the urinary tract infection symptoms or when at risk of a E. coli infection.

According to another embodiment, compounds or compositions of the invention are applied or administered after the onset of the E. coli infection.

### Combinations

SA compounds or Filipendula ulmaria extracts according to the invention and pharmaceutical formulations thereof can be administered alone or in combination with a co-agent useful in the prevention and/or treatment of urinary infections, e.g. for example a co-agent selected from D-mannose, cranberry extracts, Uvae ursi folium extracts, zinc, vitamin D3, vitamin C and antibiotic agents.

According to a further aspect is provided a formulation comprising from about 0.1 to about 50 mg of salicylic acid (SA), such as for example from about 0.5 to about 20 mg or a metabolite thereof or a mixture thereof, combined with at least one co-agent useful in the prevention and/or treatment urinary infections and at least one pharmaceutically or cosmetically acceptable carrier, diluent or excipient thereof.

According to another further aspect is provided a formulation comprising from about 0.1 to about 50 mg of salicylic acid (SA), such as for example from about 0.5 to about 20 mg or a metabolite thereof or a mixture thereof, combined with at least one co-agent useful in the prevention and/or treatment urinary infections and at least one pharmaceutically or cosmetically acceptable carrier, diluent or excipient thereof, wherein said formulation comprises an extract from species Filipendula ulmaria.

According to a particular aspect, the co-agent is selected from oenothein B and spiraeoside or a mixture thereof.

According to a particular aspect, the formulation further comprises 0.1 to about 1′000 mg of oenothein B, such as for example from about 0.5 to about 80 mg of oenothein B.

According to another particular aspect, the co-agent is an extract of Epilobium parviflorum.

According to another further aspect, a formulation according to the invention further comprising D-mannose, in particular from 500 to about 3′000 mg (e.g. 700 mg/mL).

According to another further aspect, a formulation according to the invention further comprising vitamin D3, in particular from 200 to about 2′000 UI (e.g. 500 UI).

The invention encompasses the administration of extracts according to the invention and pharmaceutical formulations thereof to an individual prior to, simultaneously or sequentially with other therapeutic/prophylactic regimens or co-agents in the prevention or treatment of urinary infections. An extract or the pharmaceutical formulation thereof that is administered simultaneously with said co-agents can be administered in the same or different composition(s) and by the same or different route(s) of administration.

The dosage administered, as single or multiple doses, to an individual will vary depending upon a variety of factors, including pharmacokinetic properties, patient conditions and characteristics (age, body weight, health, size), extent of symptoms, concurrent treatments, frequency of treatment and the effect desired.

### Patients

In an embodiment, patients according to the invention are patients suffering from a urinary infection, in particular a E. coli infection.

In another embodiment, patients according to the invention are patients at risk of suffering from urinary infection.

Patients at risk of suffering from urinary infection include patients suffering from recurrent E. coli infections, patients with suppressed immune systems, pre- and post-menopausal (oestrogen deficient) or pregnant female patients, patients with implanted catheters, frequent sexual intercourses, known previous infections episodes, repeated use of antibiotherapy, congenital malformation and diabetic patients.

According to a particular embodiment, the formulations of the invention are to be administered during the acute phase (symptomatic) of UTI and preferably for at least 5 days.

Examples illustrating the invention will be described hereinafter in a more detailed manner and by reference to the embodiments represented in the Figures.

### Examples

The following abbreviations refer respectively to the definitions below: ASE (accelerated solvent extraction), cfu/mL (colony-forming units per milliliter); DMEM (Dulbecco's Modified Eagle's medium), DMSO (dimethylsulfoxide), EDTA ((Ethylenedinitrilo)tetraacetic acid), EGF (Epidermal growth factor), FCS (foetal calf serum), HEPES (4-(2-hydroxyethyl)-1-piperazineethanesulfonic acid), PBS (Phosphate buffer saline), SDA (Sabouraud Dextrose Agar), SFM (serum free medium).

### Example 1: Effects of Salicylic Acid and Metabolites Thereof

The effects of salicylic acid (SA), CAS n° 69-72-7 (1) and two of its metabolites formed in vivo, namely 2,3-dihydroxybenzoic acid, CAS n° 303-38-8 (2) and salicyluric acid, CAS n° 487-54-7 (3) (Nelson et al. 2019, Godfrank's Toxicologic Emergencies, 11^{th} Ed. MaGraw-Hill Education; Dachineni et al. 2017, Int. J. Oncol., 51, 1661-1673) have been tested as follows:

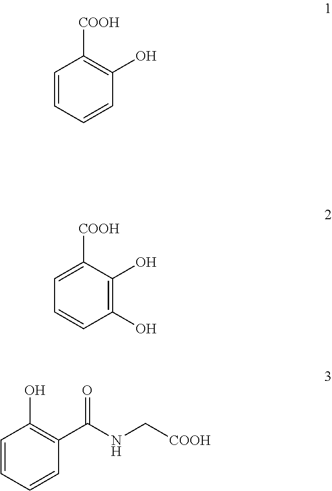 [SMILES: O=C(O)c1ccccc1O ; O=C(O)c1cccc(O)c1O ; O=C(O)CNC(=O)c1ccccc1O]

Compounds 1 to 3 were bought from Sigma. Cranberry juice extract which is widely recommended for the prophylaxis of UTIs (González de Llano et al., 2020, Molecules, 25, 352) was used as a positive control and was obtained as follow: Cranberry fruits were extracted for 1 h using a ratio plant/solvent of 1/10, followed by a filtration step and concentrated at a concentration of 5 g/L prior to storage at −20° C. Cranberry juice extract are claimed to be useful in the treatment of urinary infections (Raz et al., 2004, Clinical Infectious Diseases, 38(10), 15, 1413-1419; Wang et al., 2012, Arch Intern Med., 172(13):988-996; Gbinigie et al., 2021, Antibiotics (Basel), 10(1): 12).

In Vitro Adhesion Test (ADH)—Host-Pathogen-Interaction Assessed by In Vitro Adhesion to Human Cells Adhesion of E. coli to bladder epithelium was established in cell culture. Upon exposure to E. coli an epithelial cell detachment was observed, in a manner similar to in vivo exfoliation. The test was miniaturized by growing the epithelium cell cultures in 96-well plates instead of the classical 24-well plates. The miniaturization resulted in a higher number of measurements and reduced their variability from above 50% to below 30%, fulfilling the acceptance criteria for the reproducibility of cell-based bioassays.

The test samples were added prior to the start of the adhesion process. Bladder cells grown to confluence in 96-well plates were exposed to E. coli (50 μL of E. coli at 1.4*107 cfu/mL in each well) for 1 h. After 1 h, non-adhered E. coli were rinsed off and the adhered E. coli were detached and enumerated by plate counting colony-forming-units (cfu). Enumeration was automatized using a spiral plate count apparatus which further improved the test-variability.

The E. coli adhesion test was run in absence (given as 100% adhesion) and presence of different concentrations of the agents. The criteria of a significant reduction of the E. coli adhesion to the host, included values of <50% of normed adhesion. Human cytotoxicity measurements of the mitochondrial activity (MTT) excluded side-effects of the agents.

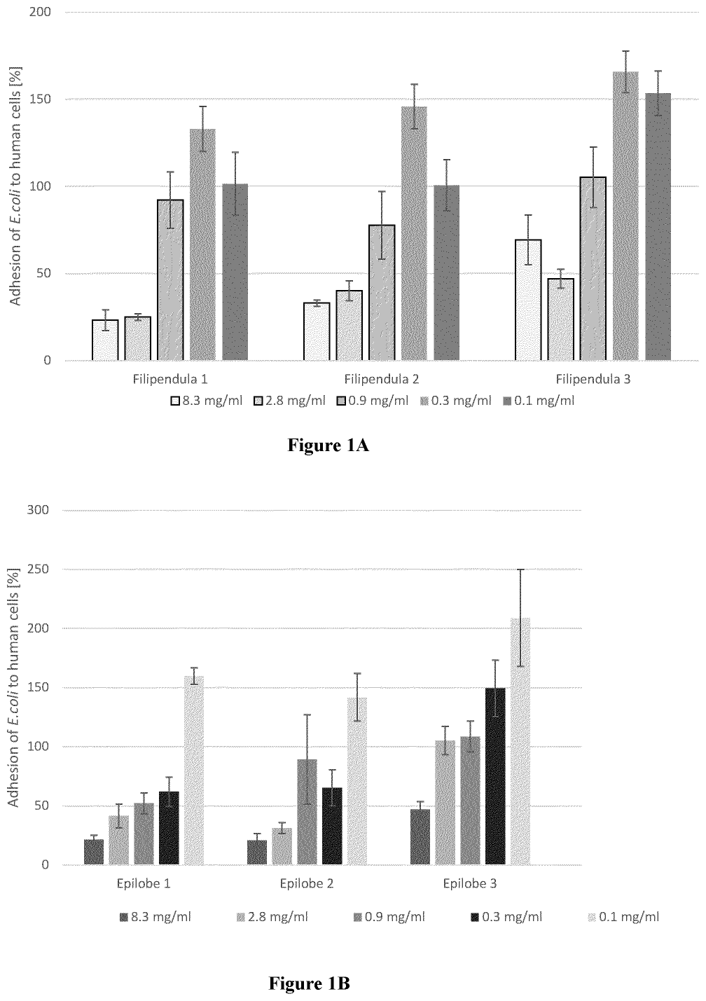

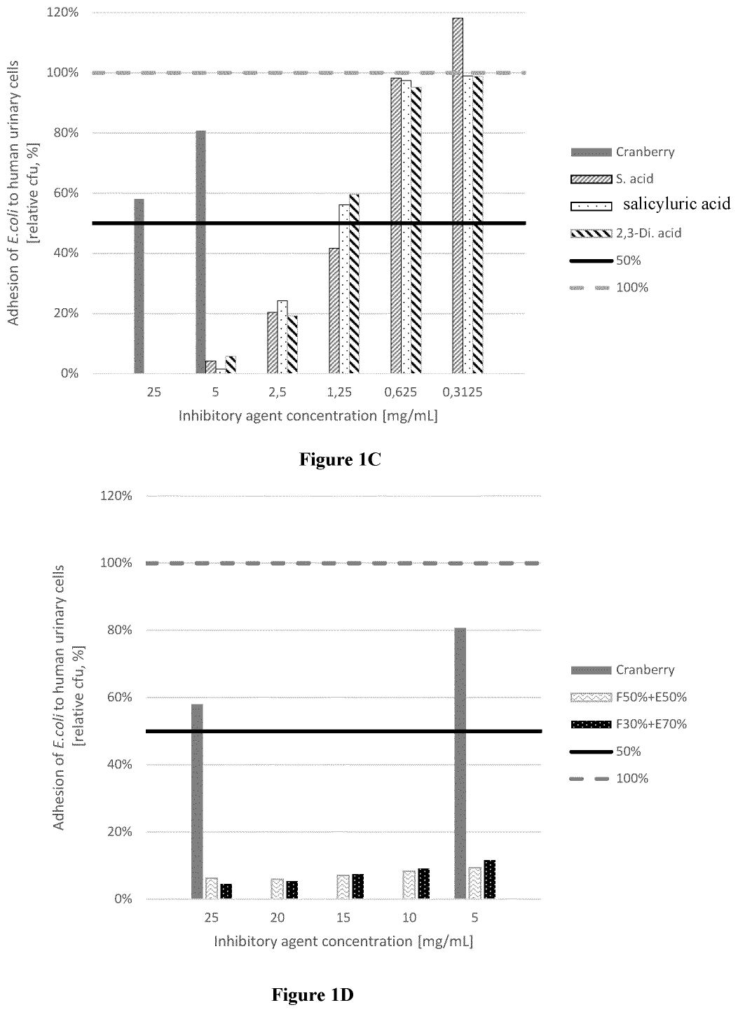

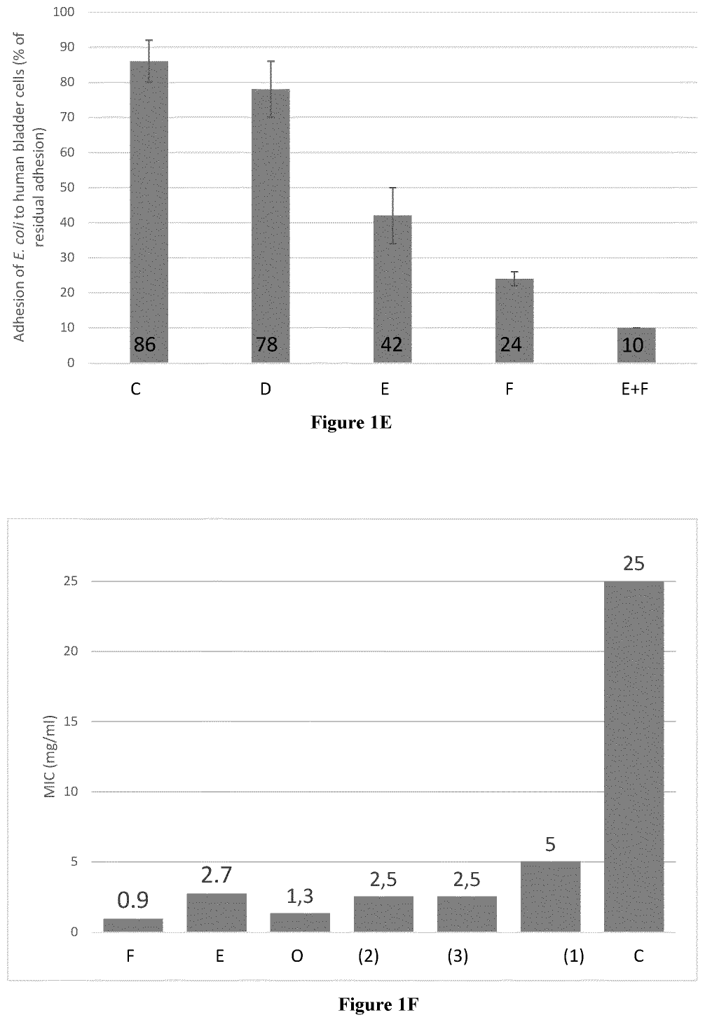

Results are presented in FIG. 1C where relative adhesion (ADH) is expressed in percent (no inhibition control=100%; buffer was used instead of the treatments). All adhesion values<50% are defined as a significant inhibition.

Those data show that salicylic acid and its metabolites induce a reduction on the adhesion for E. coli CFT073 on the urinary bladder cells. Salicylic acid presents at concentrations of 5, 2.5 and 1.25 mg/mL an adhesion reduction efficacy. Salicyluric acid shows a significant reduction at concentrations of 5 and 2.5 mg/mL. The same concentrations also show an effect for the incubation with 2,3-dihydroxybenzoic acid.

Therefore, in vitro anti-adhesion activities of salicylic acid and its two metabolites are similar.

### In Vitro Antibacterial Assay

E. coli growth in solution was measured in 96-well plates (96-well plate filled with 100 μL of diluted E. coli (2.5*10⁵ cfu/mL). Absence and presence of different concentrations of the test agent during the 24 h exponential growth determined the MIC (minimal inhibitory concentration) value. Growth is normally quantified by spectrophotometry OD_{690 nm}. However, due to the chromogenic properties of certain compounds and plant extracts, they interfered with the spectrophotometry. Therefore, adaptation was performed by E. coli enumeration by the plate-counting method (ufc counting). Inhibitory activities are presented in Table 1 below.

TABLE 1

| | CONCEN- | Observation | |
|---|---|---|---|
| | TRATION | of microbial | |
| AGENT | [mg/mL] | culture | REMARKS |
| Cranberry extract | 5 | growth | (no MIC effect) |
| | 25 | growth | |
| Salicylic acid | 0.3125 | growth | MIC at 5 mg/mL |
| | 0.625 | growth | |
| | 1.25 | growth | |
| | 2.5 | growth | |
| | 5 | no growth | |
| Salicyluric acid | 0.3125 | growth | MIC at 5 mg/mL |
| | 0.625 | growth | |
| | 1.25 | growth | |
| | 2.5 | growth * | |
| | 5 | no growth | |
| 2,3-Dihydroxy- | 0.3125 | growth | MIC at 1.25 mg/mL |
| benzoic acid | 0.625 | growth * | |
| | 1.25 | no growth | |
| | 2.5 | no growth | |
| | 5 | no growth | |

Observations made are either “growth” or “no growth” of E. coli cultures. Values under “remarks” show the concentration, at which no growth of E. coli was observed, termed MIC values. The observation “growth *” means that there was a partial inhibition measured.

Therefore, for salicylic acid and its metabolites (2 and 3), the MIC values were 5 mg/mL and 5 mg/mL 1.25 mg/mL, respectively. The cranberry extract did not show any MIC effect.

Since the concentration of salicylic acid necessary for inhibition of E. coli adhesion to 10 human bladder epithelium was of 2.5 mg/mL (=0.25 mg/100 μL) for 1 hour and the minimal inhibitory concentration on bacterial growth 5 mg/mL (=0.5 mg/100 μL) during 24 h, those data suggest that salicylic acid may be useful not only for a bactericidal direct effect but also for an inhibition of bacterial adhesion.

### In Vivo Efficacy

An in vivo test of UTI (urinary tract infection) was established and implemented for the identification of active plant extracts and compounds. CHHeNCrLmice which were age matched females were adapted to the clean, non-septic laboratory conditions. The experiment started with a 2 days nutrition enriched by glucose in order to facilitate the subsequent UTI infection. Each compound, or plant extract was then applied at various concentrations by 8 consecutive intestinal ingestions by gavage-devices over the duration of 24 h. Each ingestion comprised a volume of 100 μL solution. During these 24 h the mice were hold in metabolic cages. The urine was collected every 3 h to analyze diuresis effects. For the subsequent UTI infection*, the isolate E. coli CFT073 (commercially available at DSM, originating from human UTI) was applied trans-urethral into the bladder and incubated in vivo for a 3 h duration and free movements in normal cages. Mice were euthanized and living E. coli were enumerated in bladder homogenates (homogenization of 12 bladder for living bacteria counting (cfu)), and organ morphology were assessed by histology microscopy. *UTI Infection with 50 μL of E. coli (CFT073) containing 5×10 germs inoculated in the bladder (in another experiment, the in vivo infection was run similarly, but with slightly less germs 3.3×10⁶ or 5×10⁶ germs per inoculation).

The conversion of the doses used in animal models to Human dose (HED, Human Equivalent Dose) has been calculated according to the Guidance for Industry Estimating the Maximum Safe Starting Dose in Initial Clinical Trials for Therapeutics in Adult Healthy Volunteers published by FDA (CDER) in July 2005 (https://www.ncbi.nlm.nih.gov/pmc/articles/PMC4804402/and https://www.fda.gov/me dia/72309/download, Table 1 page 7).

### Effects on In Vivo Bladder Adhesion

In a representative experiment (n=6 mice per arm) with a bacterial concentration of 5×10⁷ cfu of E. coli applied trans-urethral into the bladder, salicylic acid (0.05 mg/100 μL) had an inhibitory effect of −60% on the bacterial infection (adhesion) in a manner similar to the positive control cranberry (2.5 mg/100 μL). In another experiment with a bacterial concentration of 3.3×10⁶ cfu applied trans-urethral into the bladder, salicylic acid (0.78 g/100 μL) had an inhibitory effect of −61.3% on the bacterial infection (adhesion) in a manner similar to the positive control cranberry (0.25 mg/100 μL).

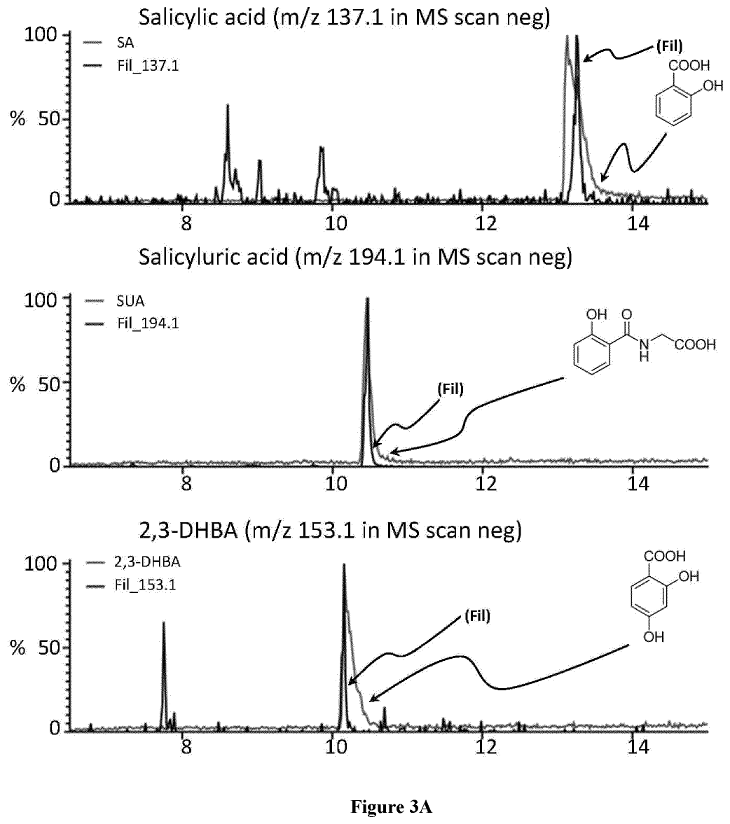

Those in vivo data show bacteriostatic and anti-adhesion effect of salicylic acid when it was applied by gavage 8 times during 24 hours at a concentration of 0.05 mg/100 μL/gavage (total of 0.4 mg/24 h). Further, since the cumulated amounts of salicylic acid and its metabolites present in the urine in those mice were 84 g for salicylic acid, 707 g for salicyluric acid and 68 g for 2,3-dihydroxybenzoic acid (FIG. 3B), it confirms that in addition to an in vitro inhibitory effect on adhesion (FIG. 1C), a direct bactericidal and anti-adhesion effect of salicylic acid is likely to play a role against E. coli urinary infection in vivo.

### Diuretic Effects

The urines were collected from control groups and from mice treated with different agents. Values of urine volume (in mg) released upon the different treatments are described in Table 2 which describes the diuresis effect of various substances and extracts tested in vivo using a mouse model by giving orally 100 μL of test substance every 3 hours for 21 h prior to inoculating 5×10⁶ E. coli in the bladder 3 h prior to ending the experiment. The experiments last overall 24 hours with urine collected and weighed every 3 hours. Results are expressed in percentage of variation compared to Negative Control (95% water, 5% glycerin), Cranberry extract as Positive Control (0.2 mg in water extract in 5% glycerin for comparison with pure substances or at the identical total concentration of extract or mixture of extracts that it is compared to) and an extract of Filipendula ulmaria and Epilobium parviforum (water extract in 5% glycerin). Each value has been calculated from the total urine collected from 6 mice.

TABLE 2

| | | % variation |
|---|---|---|
| | % variation | versus Positive |
| | versus Negative | Control |
| Test substance | Control (water) | (Cranberry) |
| Salicylic acid | 22.30% | 67.80% |
| (50 μg/100 μL) | | |
| Salicylic acid | 10.70% | 24.90% |
| (0.78 μg/100 μL) | | |
| Oenothein B | 39.90% | 57.80% |
| (18.2 μg/100 μL) | | |
| Spiraeoside | −9.10% | −4.20% |
| (68.8 μg/100 μL) | | |
| Filipendula ulmaria | 0.10% | 35.80% |
| (2.5 mg/100 μL) | | |
| Epilobium parviflorum | | −2.50% |
| (2.5 mg/100 μL) | | |
| Mixture Epilobium parviflorum + | 22.70% | 38.10% |
| Filipendula ulmaria | | |
| (5.0 + 5.0 mg/100 μL) | | |
| Mixture Epilobium parviflorum + | | 11.40% |
| Filipendula ulmaria | | |
| (2.5 + 2.5 mg/100 μL) | | |
| Mixture Epilobium parviflorum + | | 8.30% |
| Filipendula ulmaria | | |
| (0.5 + 0.5 mg/100 μL) | | |

The concentration of salicylic acid at 0.78 μg/100 μL as a pure compound tested was similar to the 1.300 concentration found in the extract of Filipendula ulmaria (see below) when used at a concentration of 0.5 mg in 100 μL. Salicylic acid given at a very low 5 concentration of 0.78 μg/100 μL induced an increase of urine volume excreted of +10.7% and +24.9% versus the negative control (water) and the positive control (cranberry extract), respectively. Salicylic acid given at a concentration of 50 μg/100 μL induced an increase of urine volume excreted of +22.3% and +67.8% versus the negative control (water) and the positive control (cranberry extract), respectively.

Therefore, those data support that salicylic acid and its 2 major metabolites have an effect on the various factors involved in urinary infection, namely bladder bacterial cell adhesion, bacterial infection and have also a diuretic beneficiary effect that would help urinary infection healing.

### Example 2: Effects of oenothein B

Oenothein B, CAS n° 104987-36-2 (4) was isolated from a water extract of Epilobium parviflorum by a combination of preparative and semi-preparative HPLC on RP18 (yield 1.3%). The compound was characterized by mass spectrometry and NMR spectroscopy. The effects of oenothein B have been tested in the same assays as described under Example 1. In vitro antibacterial effects are presented under Table 3 below and diuretic effects under Table 2 above

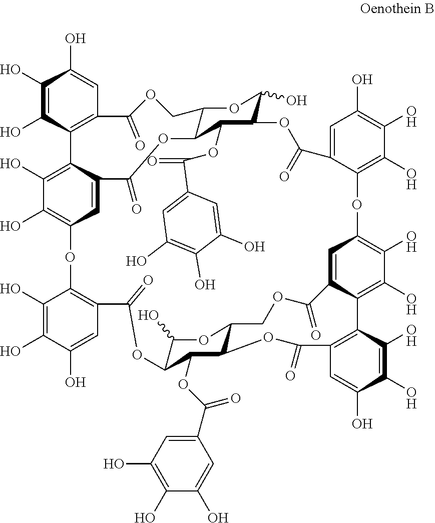

TABLE 3

| | | Observation | |
|---|---|---|---|
| | CONCENTRATION | of microbial | |
| AGENT | [mg/mL] | culture | REMARKS |
| Oenothein | 0.65 | growth | MIC at 1.3 mg/mL |
| B | 1.3 | no growth | |
| | 1.9 | no growth | |
| | 2.6 | no growth | |
| | 3.25 | no growth | |

For oenothein B the MIC was at 1.3 mg/mL.

### In Vivo Efficacy

### Effects on In Vivo Bladder Adhesion

Under the conditions of a bacterial inoculation at the concentration of 3.28×5×10⁶ cfu, the number of adhered bacteria E. coli was 75.12% lower in animals treated with oenothein B at 0.065 mg/100 mL than in the controls.

### Diuretic Effects

Oenothein B given at the concentration of 18.2 μg/100 μL induced a strong increase of urine volume excreted of +39.9% and of +57.8% versus the negative control (water) and the positive control (cranberry extract), respectively. The tested amount of oenothein B corresponds to an extract of E. parviflorum that would have been at 1.12 mg/100 μL as the concentration of oenothein B is 13% in the extract as described above.

Since oenothein B and its metabolites were not identified by LC-MS in the collected urine samples, it is suggested that other mechanisms than direct inhibition of bacterial growth in the bladder might play a role for the activity of oenothein B against E. coli urinary infections in vivo which would be indicative of a potential advantage for a combined used with SA or its metabolites.

### Example 3: Effects of Fillipendula ulmaria and Epilobium parviflorum extracts

The effects of various extracts of Filipendula ulmaria and of Epilobium parviflorum were tested as described above under the following conditions.

The following extracts were prepared:
1. Fu1 and Ep1: hot water extraction for 1 h of floral tops in a ratio plant/solvent of 1/10, filtration, measurement of dry content, addition of maltodextrine (20% of final weight), lyophilization and storage under vacuum in aluminum poaches;
2. Fu2 and Ep2: 20% EtOH extraction for 1 h of floral tops in a ratio plant/solvent of 1/10, filtration, evaporation of ethanol, measurement of dry content, addition of maltodextrine (20% of final weight), lyophilization and storage under vacuum in aluminum poaches;
3. Fu3 and Ep3: 70% EtOH extraction for 1 h of floral tops in a ratio plant/solvent of 1/10, filtration, evaporation of ethanol, measurement of dry content, addition of maltodextrine (20% of final weight), lyophilization and storage under vacuum in aluminum poaches;
4. Fu4: hot water extraction for 1 h from the aerial parts (floral tops and 60 cm of top stem) as described in a ratio plant/solvent of 1/10, filtration, evaporation of ethanol, measurement of dry content, addition of maltodextrine (20% of final weight), lyophilization and storage under vacuum in aluminum poaches.

Extracts of floral summit of Filipendula ulmaria, aerial part of Epilobium parviflorum and berries of Vaccinium oxycoccus (Cranberry) were prepared as follow: initial extraction for 1 h in hot water (70° C.) with a material to solvent ratio of 1/10. The macerate was filtered and the amount of dry material was measured. Maltodextrine DE20 was added to the filtrate with a ration of 20% of the final dry weight, prior to lyophilization. Dry powder was stored under vacuum in aluminium poaches.

### In Vitro Antibacterial Assay

The minimal inhibitory concentration (MIC) of different extracts from the different extraction procedures presented in Table 4 (uropathogenic E. coli., centrifugation of plant extracts at 14'000×g, 5 min) showed that the extraction procedure 1 (Fu1, Ep1) was pursued for further evaluation. The MIC values for Filipendula ulmaria extract alone thus are 0.9 mg/mL and for Epilobium parviflorum 2.7 mg/mL.

TABLE 4

| | MIC |
|---|---|
| | [mg/mL] |
| Filipendula ulmaria extracts | |
| Fu1 | 0.9 |
| Fu2 | 8.3 |
| Fu3 | 2.7 |
| Fu4 | 2.7 |
| Epilobium parviflorum extracts | |
| Ep1 | 2.7 |
| Ep2 | 2.7 |
| Ep3 | 25 |

E. coli growth in solution was measured in 96-well plates. Absence and presence of different concentrations of the test agent during exponential growth determined the MIC (minimal inhibitory concentration) value. E. coli enumeration was by the plate-counting method (ufc) as described above. The data are summarized under FIG. 1E showing that anti-adhesion activity is more than 2 times superior with Filipendula ulmaria extract and 8 times superior with combination of extracts of the invention compared to Cranberry extracts and D-mannose effects.

The minimal inhibitory concentration of mixtures of the hot water extracts of Filipendula ulmaria (Fu1) (F) and Epilobium parviflorum (E) are presented under Table 5 below under the same conditions as indicated above.

TABLE 5

| | | Observation | |
|---|---|---|---|
| | CONCENTRATION | of microbial | |
| AGENT | [mg/mL] | culture | REMARKS |
| F 50% + | 5 | growth | MIC at 10 mg/mL |
| E 50% | 10 | no growth | |
| | 15 | no growth | |
| | 20 | no growth | |
| | 25 | no growth | |
| F 30% + | 5 | growth | MIC at 10 mg/mL |
| E 70% | 10 | no growth | |
| | 15 | no growth | |
| | 20 | no growth | |
| | 25 | no growth | |
| | 5 | no growth | |

The MIC value for the concentration ratio of 50%+50% is at 10 mg/mL. A concentration reduction by 20% of the Filipendula ulmaria extract (% of the total mixture) and an increase in concentration by 20% of the Epilobium parviflorum extract (70% of the total mixture) results in a composition having a comparable effect when compared to the composition having a concentration ratio of F_{50%}+E_{50%}. The MIC-value is 10 mg/mL for the ratio F_{30%}+E_{70%}. Similar activities were found for extract mixtures F_{70%}+E_{30%} and F_{85%}+E_{15%}. The data are summarized under FIG. 1F showing that the compositions of the invention are active at concentrations up to 10 times inferior as Cranberry extracts.

### In Vitro Adhesion Test (ADH)

Anti-adhesive effect is measured in vitro (standard method) using centrifuged extracts (Fu1) of Filipendula ulmaria (F) and Epilobium parviflorum (E) using human epithelial bladder cells (T24 ATCC® HTB-4™ Homo sapiens urinary bladder cell lines).

FIGS. 1A and 1B supports that all tested extracts of Filipendula ulmaria (F) and Epilobium parviflorum (E), respectively have an impact on the adhesion of E. coli CFT073 to human bladder cells. FIG. 1D supports that all combined extracts of Filipendula ulmaria (F) and Epilobium parviflorum (E), at ratios of 50/50 and 30/70 tested at a total concentration of 5, 10, 15, 20 and 25 mg/mL have an impact on the adhesion of E. coli CFT073 to human bladder cells.

### In Vivo Efficacy

### Effects on In Vivo Bladder Adhesion

The in-vivo anti-adhesion effect of the various substances and extracts of the invention were tested in vivo using a mouse model as described above by giving orally 100 μL of test substance every 3 hours for 24 h prior to inoculating E. coli in the bladder 3 h before measuring E. coli in the bladder. At the end of each assay, mice are euthanized, and their bladder homogenized for E. coli measurements.

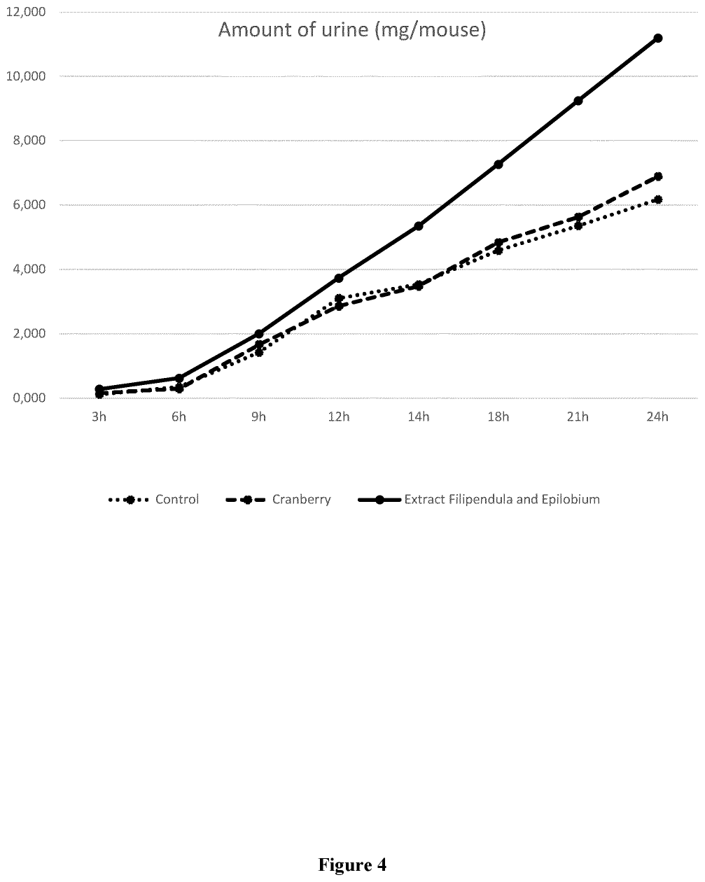

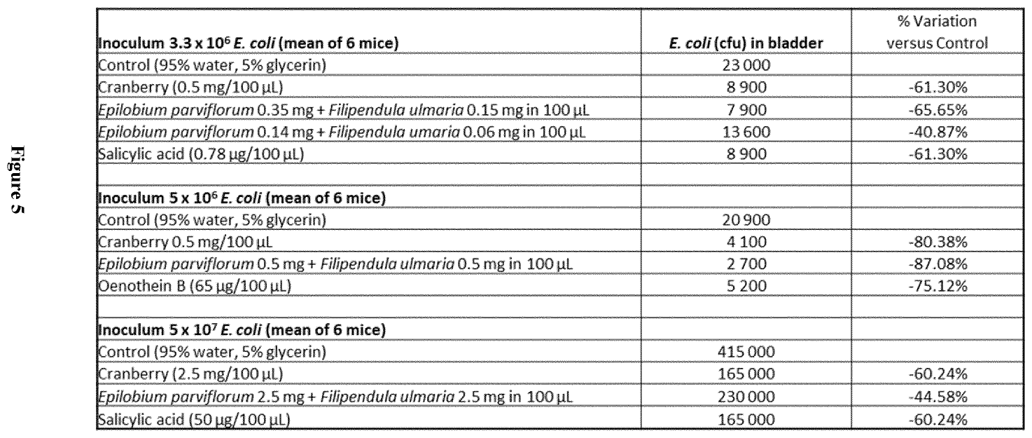

In three representative experiments (n=6 mice per arm in each experiment) with similar bacterial concentrations of 3.3×10⁶ of 5×10⁶ cfu of E. coli applied trans-urethral into the bladder, the combined extract of Filipendula ulmaria and Epilobium parviflorum showed a dose-range effect as bacteriostatic and prevention of the adhesion of E. coli compared to water control by 87.08% (F 50%+E 50%), 65.65% (F 30%+E 70%) and 40.87% (F 30%+E 70%) with concentrations of total plant extracts of 1 mg, 0.5 mg and 0.2 mg per 100 μL, respectively (FIG. 5).

This combined extracts of Filipendula ulmaria and Epilobium parviflorum at 5 mg/100 L (2.5+2.5 mg each) (F 50%+E 50%) was even effective using a higher bacterial concentration of 5×10⁷ cfu of E. coli applied trans-urethral into the bladder, in reducing the amount of E. coli by 40%, although this effect was lower than the one observed in this experiment with the Cranberry extract (60.24%).

### Diuretic Effects

The extracts were tested as described in Example 1 with results shown in Table 2. The extract of Filipendula ulmaria at a concentration of 2.5 mg/100 μL induced an increase of urine volume of 35.8% compared to the Cranberry extract given at the same concentration. No diuretic effect was measured with the extract of Epilobium parviflorum alone at a concentration of 2.5 mg/100 L.

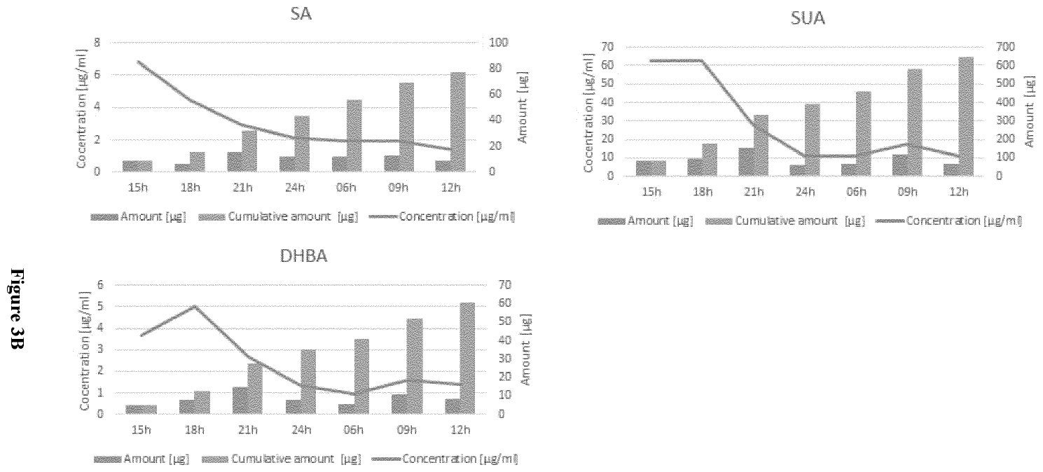

The combined extracts of Filipendula ulmaria and Epilobium parviflorum (F 50%+E 50%) showed a dose-range diuretic effect with an increase in urine volume compared to cranberry extract (5 mg/100 μl) of 38.1%, 11.4% and 8.3% with the total concentrations of the combined extracts of 10 mg, 5 mg and 1 mg per 100 μL, respectively. In another experiment, the increase in diuresis compared to controls (Cranberry extract at 5 mg/100 μL) occurs all along the 24 h of treatment as shown in FIG. 4 with a combination of extracts of Filipendula ulmaria and Epilobium parviflorum at 5 mg/100 μL (F 50%+E 50%).

Overall, one can conclude that the above effects compared to a Cranberry extract, a well-known alternative treatment for UTIs, are striking: the combination of plant extracts prevented both E. coli adhesion and growth over 8 times more than the Cranberry extract and there was a 62.4% increase in urine volume over 24 h compared to the Cranberry extract.

### Example 4: Phytochemical Characterization of Fillipendula ulmaria and Epilobium parviflorum Extracts

The extracts prepared as described in Example 3 were characterized as follows.

Polyphenolic Profile of Filipendula ulmaria and Content of Spiraeoside

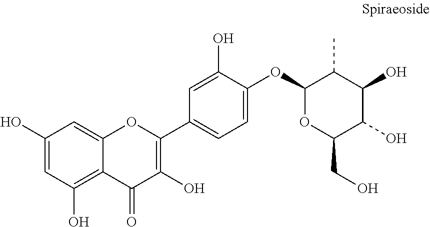 [SMILES: C[C@H]1[C@H](Oc2ccc(-c3oc4cc(O)cc(O)c4c(=O)c3O)cc2O)O[C@H](CO)[C@@H](O)[C@@H]1O]

Spiraeoside (quercetin-4′-O-glucoside) was identified by IPLC analysis (SunFire Ci8 column) as the main flavonoid glycoside in the extract of Filipendula ulmaria. In addition, quercetin, hyperoside (quercetin-3-O-galactoside) and rutin (quercetin-3-O-rutinoside) were also detected. The identities of these compounds were confirmed by chromatographic comparison with commercial authentic standards. Spiraeoside which could be used as a marker compound has been quantified in the extract by IPLC-UV analysis (SunFire C₁₈ column) using a calibration curve prepared with a commercial reference of spiraeoside (Extrasynthese) (0.125 (Cal 1), 0.063 (Cal 2), 0.031 (Cal 3), 0.016 (Cal 4), 0.008 (Cal 5), 0.004 (Cal 6) and 0.002 (Cal 7) mg/mL). The analysis was performed with three independent replicates and afforded a content of 2.8±0.3% spiraeoside in the extract of F. ulmaria.

Salicylic Derivatives in the Extract of Filipendula ulmaria

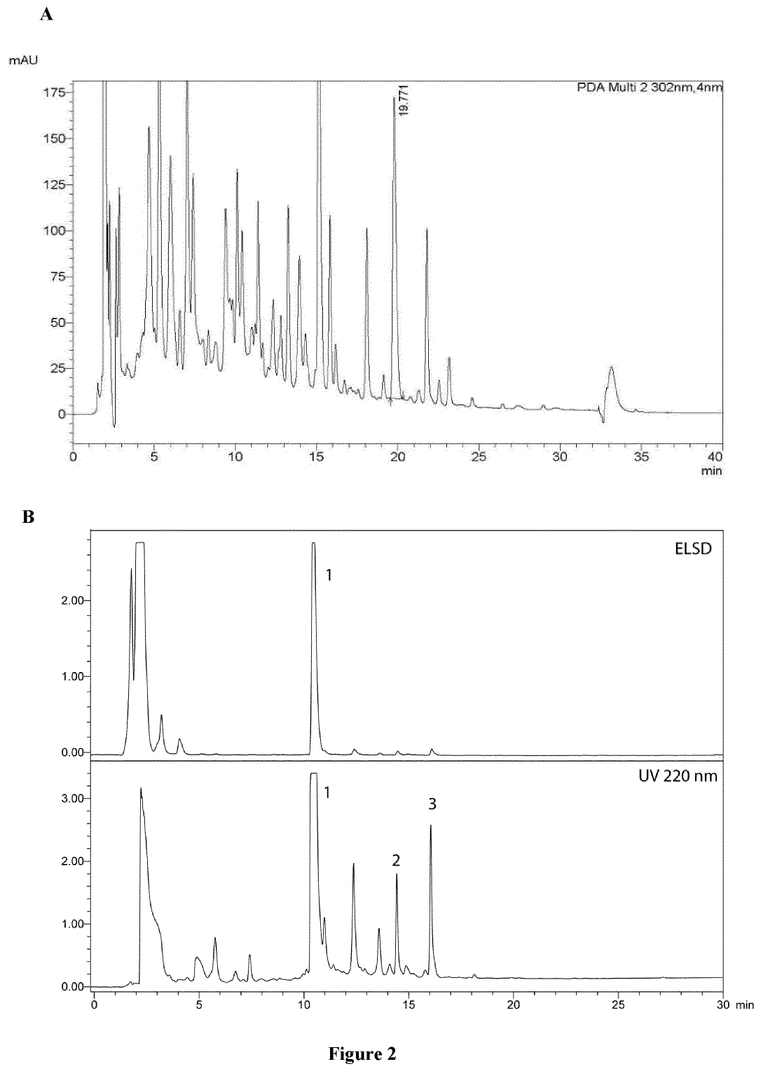

Filipendula ulmaria is known to contain among many other components, salicylic acid and derivatives thereof such as salicylic acid methyl ester and monotropitoside (Bijttebier et al 2016). Salicylic acid was quantified in the extract by IPLC-UV analysis (SunFire C₁₈ column) (FIG. 2A). A calibration curve was made with a commercial reference sample of salicylic acid (TCI Europe) (1.0 mg/mL stock solution in DMSO) with 0.25 (Cal 7), 0.125 (Cal 6), 0.063 (Cal 5), 0.031 (Cal 4), 0.016 (Cal 3), 0.008 (Cal 2) and 0.004 (Cal 1) mg/mL. Beside the content of salicylic acid, the content including salicylic acid glycosides and esters was determined after acid hydrolysis of the extract with 2N TFA for 2 h at 100° C. Experiments were performed in three independent replicates. The identity of salicylic acid was confirmed by HPLC-MS analysis and comparison with a commercial standard.

A content of 1.27±0.02% salicylic acid was determined in the extract while the content was slightly increased to 1.32±0.01% after acid hydrolysis. This indicates that most salicylic acid is present in the extract in the free form.

Polyphenolic Profile of Epilobium parviflorum and Content of oenothein B

The tannin oenothein B is by far main phenolic constituent in the Epilobium parviflorum extract. In addition, hyperoside and a myricetin-hexoside were found to be the main flavonoids. Oenothein B has been quantified by HPLC-UV analysis (SunFire C₁₈ column). A calibration curve was made with the substance isolated from Epilobium parviflorum (0.4 (Cal 4), 0.2 (Cal 3), 0.1 (Cal 2), 0.05 (Cal 1) mg/mL). A content of 13.1% was determined in the extract of Epilobium parviflorum (FIG. 2B).

### Analysis of Salicylic Acid and its Metabolites in Urines of Treated Mice.

The urines collected from control groups as described in Example 3 were compared against urines collected from mice treated with different combinations of plant extracts. The urines were analyzed using HPLC-UV/MS on a SunFire C₁₈ column column. 5% MeCN (A) and MeCN (B) both containing 0.1% of formic acid were used as mobile phase. A gradient of 0 to 100% B in 20 min was applied at a flow rate of 0.8 mL/min. MS scans were recorded in positive and negative ion modes (ESI+/−). Major additional peaks in the groups treated with a mixture of Filipendula ulmaria-Epilobium parviflorum were identified as salicylic acid (SA) (1), 2,3-dihydroxybenzoic acid (DHBA) (2), and salicyluric acid (SUA) (3). FIG. 3A compares the RT from analytical standards of salicylic acid (SA) (1) and its metabolites salicyluric acid (SUA) (2) and 2,3-dihydroxybenzoic acid (DHBA) (2), and peaks found in urine samples collected from groups treated with Filipendula extracts. The exact mass of the three molecules was confirmed using high-resolution mass spectrometry. Further, the three identified metabolites were quantified in the urine samples by MRM—with the following transitions (m z): 136.5→65.0, 136.5→92.9 (SA); 152.9→108.9, 152.9→80.9 (DHBA); 194.0→150.0, 194.0→92.9 (SUA) and pure analytical standards that were diluted in urine from negative controls for the calibration curve. FIG. 3B shows the cumulative amount and concentration of salicylic acid (SA), salicyluric acid (SUA) and dihydroxybenzoic acid (DHBA) in urine samples collected over 21 hours from group treated with 40 mg Filipendula extract (administered in 8 doses at 3 h time intervals).

The amounts of SA, SUA et DHBA were quantified in the urine of mice after absorption of an extract of Filipendula ulmaria containing 1.3% of SA at a total dose of 40 mg divided in 8 equal doses over 24 h as described above (Example 3, in-vivo efficacy studies). It was found that 41% of SA that has been ingested is found in the urines in the form of: SA (5.4%), SUA (32.0%) and DHBA (3.9%) (Total 41%).

Therefore, those experiments confirm that by administering and extract of Filipendula ulmaria orally, it is possible to measure the presence of SA and its metabolites at the site of action with a recovery of 41% of the amount ingested found under SA, SUA and DHBA.

Altogether, the in vitro data support the beneficial effects of SA and its metabolites formed in vivo after SA ingestion against E. coli urinary infections since it has not only an effect against the adhesion of the bacteria to the bladder cells but also a direct bactericidal effect. It is further suggested that by the data that oenothein B has an in vivo activity against E. coli urinary infections through another mechanism than a direct inhibition of bacterial growth in the bladder and therefore, the activity of SA and its metabolites would be complementary in an attempt to combat E. coli urinary infections. Further, the present data also show that the administration of SA and of its derivatives, 2,3-dihydroxybenzoic acid, and salicyluric acid could be successfully achieved through the administration of Filipendula ulmaria extract, preferably an aqueous extract. Based on the active doses in mice, one could reasonable extrapolate (FDA (CDER) in July 2005, supra: https://www.fda.gov/media/72309) that the use of a composition comprising from about 0.25 to 2.6 mg of SA would be beneficial. This dose might be achieved by an extract of Filipendula ulmaria from about 10 to about 200 mg dry matter weight, in particular from about 20 about 100 mg.

SA might be advantageously combined with oenothein B, in particular from about 1.3 to about 65 mg of oenothein B.

Furthermore, administration of oenothein B could be achieved through the administration of Epilobium parviflorum extract, preferably an aqueous extract. The above dose of oenothein B might be achieved by an extract of Epilobium parviflorum from about 10 to about 1'000 mg dry matter weight, in particular from about 10 to 500 mg.

Finally, FIGS. 1A, 1B and 1C further support that combinations of extracts of Filipendula ulmaria and Epilobium parviflorum according to the invention have a beneficial effect against E. coli infections and since those contain identified active principles that have been demonstrated to have complementary activities, it is expected that such combinations would have advantageous multi-targeting effects against E. coli urinary infections and are better than each extract alone.

### Example 5: Effects on Inflammation

The effect of compositions of the invention can be assessed on CACO-2 cells on which an inflammatory response has been triggered by TNF-α. The anti-antiflammatory effect can be measured through the quantification of the amount of IL-6 released in response to TNF-α (Vitkus et al. 1998, In vitro cellular and Developmental Biology, 34, 660-664).

### Example 6: Effects of Combinations of Extracts in Recurrent UTIs

The effect of compositions of the invention is assessed on adult women (15 women aged 18 to 65 years of age) who have recurrent UTIs (more than 3 repetitions per year). Tablets incorporating a mixture of the extracts of the invention are prepared by standard methods using lyophilized hot water extracts of Flipendula ulmaria and Epilobium parviflorum as described above (Example 3) with 1 tablet containing 132 mg of a mixture of two plant extracts described above (Flipendula ulmaria, 23 mg, Epilobium parviflorum, 109 mg). The mixture corresponds to F_{17%}+E_{83%}.

1 Tablet contains also 700 mg of D-mannose, 80 mf of FOS, 1.25 mg of Zinc gluconate and 500 UI of Vitamin D3.

As soon as UTI symptoms appeared, women were asked to take a tablet 20 minutes before meals for 5 days according to the following schedule: 2 tablets in the morning in a single dose and 2 in the evening in a single dose, even if symptoms disappeared before the end of the treatment. If the infection required antibiotics, patients could continue taking the regiment since no interaction or contraindications are known to date. Two Patients who took antibiotics during the assay were excluded of the statistical analysis that was performed with 15 patients.

The results were assessed through a questionnaire to the treated women:
1. On a scale of 0 to 10 (0 none, 10 severe), how severe would you say your usual urinary symptoms were during your last two urinary infection before taking the treatment and how severe would you say your experienced urinary symptoms were whilst and after taking the treatment.
A: Pain during urination
B: Frequent need to urinate
C: Heaviness/pain in the bladder
2. How long (days hours) did your last two UTIs last
3. How long (days hours) did the urinary tract infection last during the treatment
4. Did you need to take antibiotics concomitantly with the treatment?
Yes/No (If no, please skip to question 5)
A. If yes, which antibiotic/duration of treatment:
B. Did you experience an improvement in the intensity or duration of symptoms when combining the treatment and the antibiotic

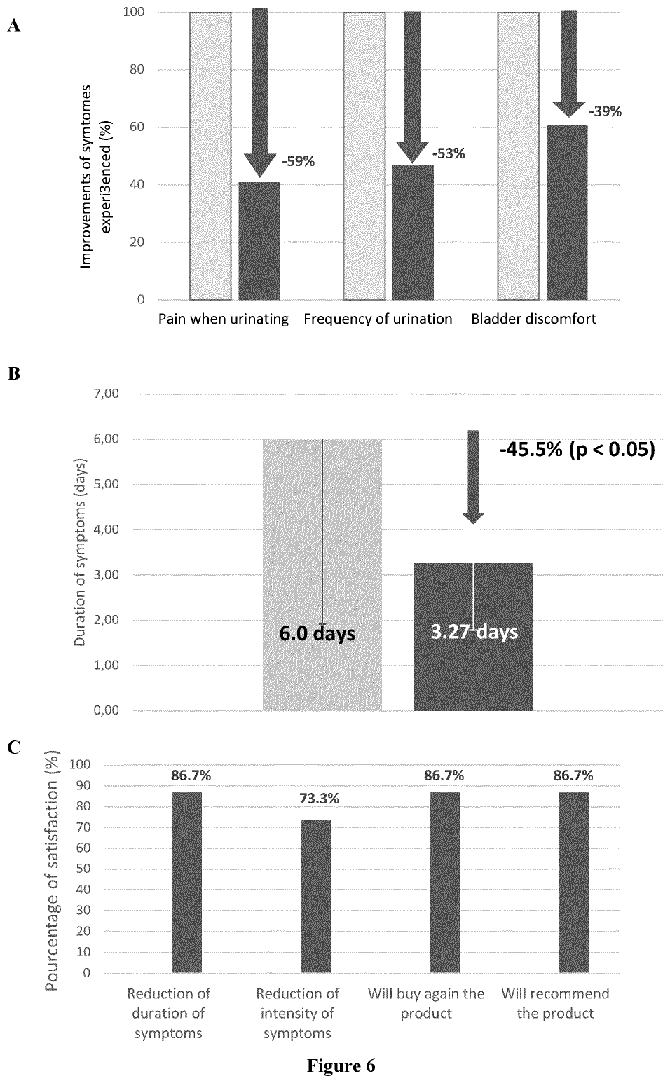

The results are shown under FIG. 6 which indicate that pain when urinating was reduced by 59%, frequency of urination decreased by 53%, pain and bladder discomfort were reduced by 39%. The duration of symptoms was significantly reduced by 45.5% (p<0.05) from the average value of 6 days (mean value without treatment, individual values from 2 to 14 days) to 3.27 days with treatment (individual values from 1 to 5 days). 86.7% of the women in the study were satisfied with the reduction of the duration of symptoms, 73.3% were satisfied with the reduction of symptoms, 86.7% will buy again the product in case of the appearance of another episode of UTI and 86.7% of them declared that they would recommend this treatment to friends suffering from UTIs.

These results are extremely promising in a context of growing antibiotic resistance and a strong need for alternative options for the prevention and treatment of UTIs.

1-21. (canceled)

22. An oral formulation comprising salicylic acid (SA) or a metabolite thereof or a mixture thereof, wherein said formulation comprises from about 0.75 μg to 2.6 mg of SA and at least one pharmaceutically or cosmetically acceptable carrier, diluent or excipient thereof, wherein said metabolite is a metabolite formed in vivo after ingestion of salicylic acid, for use for the prevention, repression and/or treatment of a urinary infection.

23. The oral formulation according to claim 22, wherein said formulation comprises from about 1 to about 550 mg dry matter weight of a Filipendula ulmaria extract and at least one pharmaceutically or cosmetically acceptable carrier, diluent or excipient thereof.

24. The oral formulation according to claim 23, wherein the extract is an alcoholic or hydroalcoholic extract.

25. The oral formulation according to claim 23, wherein the extract is an extract of the floral tops of Filipendula ulmaria.

26. The oral formulation according to claim 22, wherein said formulation further comprises oenothein B.

27. The oral formulation according to claim 26, wherein said formulation comprises from about 15 μg to about 65 mg of oenothein B.

28. The oral formulation according to claim 26, wherein said formulation comprises an extract of Epilobium, in particular Epilobium parviflorum.

29. The oral formulation according to claim 28 comprising 10 to about 1000 mg dry matter weight of a Epilobium parviflorum extract.

30. The oral formulation according to claim 22, wherein said formulation further comprises at least one ingredient selected from D-mannose and Vitamin D3.

31. A formulation comprising salicylic acid (SA) or a metabolite thereof or a mixture thereof, said formulation further comprising at least one co-agent useful in the prevention and/or treatment urinary infections and at least one pharmaceutically or cosmetically acceptable carrier, diluent or excipient thereof, wherein said metabolite is a metabolite formed in vivo after ingestion of salicylic acid.

32. The formulation according to claim 31, wherein said formulation comprises from about 1 to about 550 mg dry matter weight of a Filipendula ulmaria extract and at least one pharmaceutically or cosmetically acceptable carrier, diluent or excipient thereof.

33. The formulation according to claim 31, wherein said formulation further comprises oenothein B.

34. A method for preventing and/or treating a urinary infection in a subject, said method comprising administering by oral route in a subject in need thereof, an effective amount of salicylic acid (SA) or a metabolite thereof, or a mixture thereof or pharmaceutical compositions thereof, wherein said metabolite is a metabolite formed in vivo after ingestion of salicylic acid.

35. The method according to claim 34, wherein said metabolite is selected from 2,3-dihydroxybenzoic acid and salicyluric acid.

36. The method according to claim 34, wherein said salicylic acid (SA) or a metabolite thereof is administered in combination with oenothein B.

37. The method according to claim 34, wherein SA or said metabolite thereof is provided in a composition comprising from about 0.75 μg to 2.6 mg of SA.

38. The method according to claim 34, wherein said salicylic acid (SA) is in a form of a Filipendula ulmaria extract.

39. The method according to claim 34, wherein the urinary tract infection is a E. coli infection.

40. The method according to claim 34, wherein said salicylic acid (SA) or said metabolite thereof is administered in combination with oenothein B and oenothein B is provided in a composition comprising from about 15 μg to about 65 mg of oenothein B.

41. The method according to claim 34, wherein said salicylic acid (SA) or a metabolite thereof is administered in combination with an Epilobium parviflorum extract.

42. The method according to claim 34, wherein an oral formulation comprising salicylic acid (SA) or a metabolite thereof or a mixture thereof, wherein said formulation comprises from about 0.75 μg to 2.6 mg of SA and at least one pharmaceutically or cosmetically acceptable carrier, diluent or excipient thereof, wherein said metabolite is a metabolite formed in vivo after ingestion of salicylic acid is administered.

43. The method according to claim 34, wherein the formulation comprises from about 1 to about 550 mg dry matter weight of a Filipendula ulmaria extract and at least one pharmaceutically or cosmetically acceptable carrier, diluent or excipient thereof.
